(19) United States
(12) Patent Application Publication
(10) Pub. No.: US 2010/0098721 A1
(43) Pub. Date: Apr. 22, 2010

(54) METHODS AND COMPOSITIONS FOR PREPARING A UNIVERSAL INFLUENZA VACCINE

(75) Inventors: Jonathan A. McCullers, Memphis, TN (US); Victor C. Huber, Dakota Dunes, SD (US); Karen S. Slobod, Somerville, MA (US); Julia L. Hurwitz, Germantown, TN (US)

Correspondence Address: ALSTON AND BIRD LLP;ST. JUDE CHILDREN'S RESEARCH HOSPITAL, BANK OF AMERICA PLAZA, 101 SOUTH TRYON STREET, SUITE 4000, CHARLOTTE, NC, 28280-4000, US

(73) Assignee: St. Jude Children's Reseach Hospital, Memphis, TN (US)

(21) Appl. No.: 12/445,551

(22) PCT Filed: Oct. 17, 2007
(86) PCT No.: PCT/US07/81583

§ 371 Date: Nov. 24, 2009

Related U.S. Application Data
(60) Provisional application No. 60/852,464, filed on Oct. 18, 2006.

(51) Int. Cl. A61K 39/295; A61K 39/00; A61K 39/145

(52) U.S. Cl. 424/202.1; 424/184.1; 424/209.1

(57) ABSTRACT

Compositions and methods for preventing and treating influenza virus infections are provided. Compositions include novel immunogenic compositions having at least one vector capable of expressing an antigenic component from four or more influenza viruses representative of the antigenic diversity of a target population of influenza virus strains. Compositions further include novel immunogenic compositions having at least four vectors, each vector capable of expressing a single antigenic component representative of the antigenic diversity of a target population of influenza virus strains. In other aspects of the invention, the immunogenic compositions also include one or more viral proteins (or antigenic portions thereof), or one or more live attenuated viruses, derived from the target population of influenza virus strains. The invention further provides methods for making a multi-valent influenza vaccine and methods for inducing an immune response in a subject against influenza viruses.

### FIELD OF THE INVENTION

The present invention relates to the field of immunogenic compositions for preventing or treating influenza infection.

### BACKGROUND OF THE INVENTION

Influenza, more commonly known as the flu, is an acute, viral infection of the respiratory tract that accounts for significant levels of morbidity and mortality. The Centers for Disease Control and Prevention estimates that approximately 10% to 20% of the general population of the United States are afflicted with the flu annually, resulting in approximately 36,000 influenza-associated deaths and more than 200,000 hospitalizations (Thompson, W W, JAMA 289:179-86, 2003). Uncomplicated influenza is characterized by the abrupt onset of constitutional (e.g., fever, myalgia, headache, severe malaise) and respiratory (e.g., nonproductive cough, sore throat and rhinitis) symptoms. The flu is particularly dangerous for young children, elderly individuals, and for chronically ill patients. Direct costs associated with providing medical care for individuals afflicted with influenza have been estimated at between $1 billion and $3 billion annually.

Influenza viruses cause disease in all age groups. Children experience the highest incidence of influenza disease, two to ten times that seen in adults, and are a common source of its spread. However, rates of serious illness are highest among individuals who are 65 years of age or older, and people of any age who have medical conditions that place them at high risk for developing complications from the flu. Although healthy adults under the age of 65 are at a comparatively low risk for severe illness, the flu can result in substantial mortality, numerous health-provider visits and lost workdays. Estimates of annual indirect costs resulting from absenteeism from school and work and reduced productivity range from $10 to $15 billion.

Several properties contribute to the epidemiological success of the influenza viruses. First, they are spread easily from person to person by aerosol (droplet infection). Second, small changes in influenza virus antigens are frequent (antigenic drift) so that the virus readily escapes protective immunity induced by a previous exposure to a different variant of the virus. Third, new strains of influenza virus can be easily generated by reassortment or mixing of genetic material between different strains (antigenic shift). In the case of influenza A virus, such mixing can occur between subtypes or strains that affect different species. The 1957 and 1968 pandemics are thought to have been caused by a hybrid strain of virus derived from reassortment between a swine and a human influenza A virus.

Despite intensive efforts, there is still no satisfactory therapy for influenza virus infection and existing vaccines are limited in value in part because of the properties of antigenic shift and drift described above. For these reasons, global surveillance of influenza A virus has been underway for many years, and the National Institutes of Health designates it as one of the top priority pathogens for biodefense. Although current vaccines based upon inactivated virus are able to prevent illness in approximately 70-80% of healthy individuals under age 65, this percentage is far lower in the elderly or immunocompromised. In addition, the expense and potential side effects associated with vaccine administration make this approach less than optimal. Although the four antiviral drugs currently approved in the United States for treatment and/or prophylaxis of influenza are helpful, their use is limited due to concerns about side effects, compliance, and emergence of resistant strains. Therefore, there remains a need for the development of effective therapies for the treatment and prevention of influenza infection.

### SUMMARY OF THE INVENTION

The present invention is directed to compositions and methods for the treatment and prevention of influenza infections. Compositions of the invention include antigenic components of influenza viruses useful in vaccines. The antigenic components are selected to elicit a cross-reactive response, including the production of antibodies and/or a T cell response, against viral proteins from influenza virus strains across a target population of viruses, and thus provide immunity to a number of influenza virus strains within the target population of viruses. In one embodiment, compositions of the invention include an immunogenic composition having at least one vector capable of expressing an antigenic component from four or more influenza viruses representative of the antigenic diversity of a target population of influenza virus strains. Alternatively, compositions also include an immunogenic composition having at least four vectors, each vector capable of expressing a single antigenic component representative of the antigenic diversity of a target population of influenza virus strains. In specific, non-limiting examples, the antigenic component includes hemagglutinin proteins, or antigenic portions thereof. Exemplary vectors include, but are not limited to, plasmids, viral vectors and whole viruses.

In other aspects of the invention, the immunogenic compositions also include one or more viral proteins, or antigenic portions thereof, derived from the target population of influenza virus strains, and/or one or more live attenuated viruses derived from the target population of influenza virus strains.

Methods for making a multi-valent influenza vaccine are further provided. These methods include (i) determining the range of antigenic diversity among an antigenic component within a target population of influenza virus strains; (ii) selecting the antigenic component from four or more influenza viruses representative of the antigenic diversity of the target population of influenza virus strains, where cross-protective immunity to the antigenic component provides immunity to multiple viruses within the target population; and (iii) preparing an immunogenic composition having at least one vector capable of expressing the selected antigenic components, where the vector includes regulatory sequences operably linked to antigenic component-encoding sequences. In one embodiment, the immunogenic composition includes four or more vectors, each capable of expressing a single antigenic component. In specific, non-limiting examples, the antigenic component includes hemagglutinin proteins, or antigenic portions thereof.

Methods for inducing an immune response in a subject against influenza viruses are further provided. These methods include administering to a subject a therapeutically effective amount of an immunogenic composition as disclosed herein.

### BRIEF DESCRIPTION OF THE DRAWINGS

FIGS. 1A-E illustrate serum antibody reactivity after vaccination with influenza hemagglutinin (HA) DNA. Sera taken 14 days after the third exposure of mice to influenza HAs via DNA vaccine were analyzed for expression of influenza-specific IgG1 antibodies. Vaccination with the individual HAs (HK68, Vic75 or Len86; FIGS. 1B-D, respectively) resulted in reactivity with viruses expressing the individual HAs and some reactivity with related outside strains. When all three HAs (HK/Vic/Len; FIG. 1E) were delivered simultaneously, reactivity against all three vaccinating HAs was seen, as well as reactivity with additional outside strains not included in the vaccine. This reactivity was seen in a non-competitive manner for the three HAs delivered in the assay. The viruses against which the sera were analyzed are displayed in order of year of isolation; the figure in the bottom left shows a phylogenetic tree for the different strains. The 25^{th}-75^{th} percentiles of the antibody titers are represented by box-plots, with the horizontal bar indicating the mean value. Error bars indicate the standard deviation of the measurements. ND=not done.

FIGS. 2A-E illustrate lung viral titers after influenza virus challenge. Mice (n=4 per group) vaccinated via gene gun with DNA plasmids expressing the HA of HK68 (FIG. 2B), Vic75 (FIG. 2C), Len86 (FIG. 2D), all three (HK/Vic/Len; FIG. 2E), or mock-vaccinated with empty vector (pHW2000; FIG. 2A) were challenged with 100 MID₅₀ of HK68, Vic75, Len86, or a heterologous virus most closely related to Len86 (Mem86). Symbols represent the lung viral titer of an individual mouse. Vaccination with all three constructs resulted in the broadest and most robust protective immunity. The viruses against which the sera were analyzed are displayed in order of year of isolation; the figure in the bottom left shows a phylogenetic tree for the different strains.

FIGS. 3A-D demonstrate serum antibody reactivity after vaccination with influenza virus ts mutants. Sera taken 21 days after exposure of mice to influenza HA in the form of the influenza ts virus mutants (primary response to HA) were determined using the HI assay. Mice were mock-primed with empty vector DNA. Vaccination with the individual HAs (HKₜₛ, Vicₜₛ or Lenₜₛ; FIGS. 3A-C, respectively) resulted in reactivity with viruses expressing the individual HAs and some reactivity with related outside strains. When all three HAs (HKₜₛ/Vicₜₛ/Lenₜₛ; FIG. 3D) were delivered simultaneously, reactivity against all three of the vaccinating HAs was seen. The viruses against which the sera were analyzed are displayed in order of year of isolation; the figure in the bottom left shows a phylogenetic tree for the different strains. The 25^{th}-75^{th} percentiles of the antibody titers are represented by box-plots with the horizontal bar indicating the mean value. Error bars indicate the standard deviation of the measurements. ND=not done.

FIGS. 4A-E illustrate lung viral titers after influenza virus challenge. Mice (n=4-5 per group) were mock-primed with empty vector (pHW2000; FIG. 4A) or primed with DNA expressing HK68, Vic75 and Len86 HAs and then vaccinated with live attenuated influenza viruses expressing the HA of HK68 (HKₜₛ; FIG. 4B), Vic75 (Vicₜₛ; FIG. 4C), Len86 (Lenₜₛ; FIG. 4D), or all three (HKₜₛNicₜₛ/Lenₜₛ; FIG. 4E). They were then challenged with 100 MID₅₀ of HK68, Vic75, Len86 (FIGS. 4B-E), or a heterologous virus most closely related to Len86 (Mem86; FIG. 4A). Symbols represent the lung viral titer of an individual mouse. Vaccination with any live attenuated virus was sufficient to induce sterilizing immunity against the heterologous strain Mem86 (FIG. 4A) as well as homologous viruses. Prime-boost vaccination with all three strains generated sterilizing immunity against all viruses tested (FIGS. 4B-E); competition among strains was not observed. The viruses against which the sera were analyzed are displayed in order of year of isolation; the figure in the bottom left shows a phylogenetic tree for the different strains.

FIGS. 5A-D illustrate serum antibody reactivity after vaccination with influenza HA DNA and ts mutants. Sera taken 21 days after exposure of mice to influenza HA in the form of the influenza ts virus mutants (secondary response to HA) were analyzed using the HI assay. Mice had been primed by DNA vaccination with HAs from HK68, VI75 and LE86. Vaccination with the individual HAs (HKₜₛ, Vicₜₛ or Lenₜₛ; FIGS. 5A-C, respectively) resulted in reactivity with viruses expressing the individual HAs and some reactivity with outside strains (the breadth of the response to the ts mutants was greater and the mean titers were higher following DNA priming with HA compared to the empty vector). When all three HAs were delivered simultaneously (HKₜₛNicₜₛ/Lenₜₛ; FIG. 5D), reactivity against all three of the vaccinating HAs was seen, and in this case the competition seen when the ts mutants were delivered alone was not seen. The viruses against which the sera were analyzed are displayed in order of year of isolation; the figure in the bottom left shows a phylogenetic tree for the different strains. The 25^{th}-75^{th} percentiles of the antibody titers are represented by box-plots with the horizontal bar indicating the mean value. Error bars indicate the standard deviation of the measurements. ND=not done.

### DETAILED DESCRIPTION OF THE INVENTION

The present invention provides compositions and methods for the treatment and prevention of viral influenza infections. Compositions of the invention include antigenic components of influenza viruses useful in vaccines. In some embodiments, compositions of the invention include an immunogenic composition having at least one vector capable of expressing an antigenic component from four or more influenza viruses representative of the antigenic diversity of a target population of influenza virus strains. In other embodiments, compositions also include an immunogenic composition having at least four vectors, each vector capable of expressing a single antigenic component representative of the antigenic diversity of a target population of influenza virus strains. In specific, non-limiting examples, the antigenic component includes hemagglutinin proteins, or antigenic portions thereof.

In one embodiment, the target population of influenza virus strains includes influenza virus isolates of an individual type. In another embodiment, the target population of influenza virus strains includes influenza virus isolates of an individual subtype. In yet another embodiment, the target population of influenza virus strains includes influenza virus isolates of a geographic region. Exemplary influenza virus strains include, but are not limited to, type B influenza, subtype H3N2 type A influenza, subtype H1N1 type A influenza, subtype H5N1 type A influenza, subtype H9N2 type A influenza, subtype H6N1 type A influenza, and subtype H7N7 type A influenza.

The term “antigenic component(s)” in reference to influenza viruses includes, for example, viral transmembrane and surface proteins and glycoproteins, such as HA and neuraminidase (NA). The term also encompasses antigenic portions of viral proteins and glycoproteins (including conserved T cell epitopes).

The immunogenic compositions disclosed herein are designed to elicit both T cell and B cell responses against antigens representative of the antigenic diversity of a target population of influenza virus strains. In specific embodiments, the antigenic component-encoding sequences are contained in plasmid constructs, which can be used as a DNA vaccine, as well as a source of recombinant protein for subsequent protein boosts.

Methods for inducing an immune response in a subject against influenza viruses are further provided. These methods include administering to a subject a therapeutically effective amount of the immunogenic compositions disclosed herein.

Influenza viruses belong to the Orthomyxoviridae family and are enveloped, negative-stranded RNA viruses. They are classified as influenza types A, B, and C, of which influenza A is the most pathogenic and is believed to be the only type able to undergo reassortment within animal strains. Influenza types A, B, and C can be distinguished by differences in their nucleoprotein and matrix proteins. Influenza A subtypes are defined by variation in their HA and NA genes and usually distinguished by antibodies that bind to the corresponding proteins.

The influenza A viral genome consists of eleven genes distributed in eight RNA segments. The genes encode eleven proteins, including the envelope glycoproteins hemagglutinin and neuraminidase. Influenza A virus classification is based on the hemagglutinin (H1-H16) and neuraminidase (N1-N9) genes. World Health Organization nomenclature defines each virus strain by its animal host of origin (specified unless human), geographical origin, strain number, year of isolation, and antigenic description of HA and NA. See Julkunen et al., Cytokine and Growth Factor Reviews, 12:171-80, 2001 for further details regarding the influenza A virus and its molecular pathogenesis. The organization of the influenza B viral genome is similar to that of influenza A, while the influenza C viral genome contains seven RNA segments and lacks NA.

Genetic variation in influenza A occurs by two primary mechanisms. Genetic drift, which occurs via point mutations (which often occur at antigenically significant positions due to selective pressure from host immune responses), and genetic shift (also referred to as reassortment), involving substitution of a whole viral genome segment of one subtype by that of another. Many different types of animal species, including humans, swine, birds, horses, aquatic mammals, and others, can become infected with influenza A viruses. Some influenza A viruses are restricted to a particular species and will not normally infect a different species. However, some influenza A viruses may infect several different animal species, principally birds, swine, and humans. This ability is considered to be responsible for major antigenic shifts in influenza A virus. When two (or more) different viruses reproduce in the same host cell simultaneously, the genes of the two strains may “mix,” resulting in a new virus with a unique combination of RNA segments. This process is called genetic reassortment.

In one embodiment, the present invention pertains to immunogenic compositions useful as a vaccine. As noted, the compositions include antigenic components that are selected to elicit a cross-reactive response, including the production of antibodies and/or a T cell response, against viral proteins from influenza virus strains across a target population of viruses, thus providing immunity in a subject to a number of influenza virus strains within the target population of viruses. The term “subject,” as used herein, refers to an individual susceptible to infection with a virus, for example, an influenza virus. The term includes birds and mammals, for example, domesticated birds and mammals (such as poultry and swine), wild animals (e.g., migratory birds), non-human primates, and humans.

By “immunogenic composition” is intended a composition useful for stimulating or eliciting a specific immune response (or immunogenic response) in a subject, such as a cell-mediated immune response, a humoral immune response, or both (which can originate from naïve or memory cells). An immunogenic composition can include, for example, an antigenic component-expressing vector (e.g., an HA-expressing vector) and/or an antigenic component (e.g., an HA protein). In some embodiments, the immunogenic response is protective or provides protective immunity, in that it enables the subject to better resist infection with or disease progression from the influenza virus against which the composition is directed. One specific example of a type of immunogenic composition is a vaccine. In some embodiments, a therapeutically effective amount of an immunogenic composition is administered to a subject. A “therapeutically effective amount” of an immunogenic composition is an amount which, when administered to a subject, is sufficient to achieve a desired effect in a subject being treated with that composition. For example, this may be the amount of an immunogenic composition useful in increasing resistance to, preventing, ameliorating, and/or treating infection and disease caused by an influenza virus in a subject. Ideally, a therapeutically effective amount of an immunogenic composition is an amount sufficient to increase resistance to, prevent, ameliorate, and/or treat infection and disease caused by an influenza virus in a subject without causing a substantial cytotoxic effect in the subject. The effective amount of an immunogenic composition useful for increasing resistance to, preventing, ameliorating, and/or treating infection and disease caused by an influenza virus in a subject will depend on the subject being treated, the severity of the affliction, and the manner of administration of the immunogenic composition. Responses to a therapeutically effective amount of an immunogenic composition can include, for instance, a cell-mediated immune response, a humoral immune response, or both (which can originate from naïve or memory cells).

As described herein, compositions of the invention include immunogenic compositions having one or more vectors. As used herein, the term “vector” refers to an agent capable of mediating entry of (e.g., transferring, transporting, etc.) a nucleic acid molecule (e.g., an antigenic component-encoding sequence, such as an HA- or HA fragment-encoding sequence) into a cell. Transferred nucleic acid molecules can include sequences encoding antigenic components (e.g., HA proteins and/or antigenic portions thereof), including recombinant sequences expressing one or more antigenic component epitopes (e.g., one or more HA epitopes) from one or more viruses. The transferred nucleic acid is generally linked to (e.g., inserted into) the vector (e.g., a vector nucleic acid molecule). A vector can include sequences that direct autonomous replication, or can include sequences sufficient to allow integration into host cell DNA. Useful vectors include, for example, whole viruses, plasmids (typically DNA molecules), cosmids, yeast artificial chromosomes, and bacteriophage or eukaryotic viral DNA. Such vectors can be introduced into a variety of hosts including somatic cells, and simple or complex organisms, such as bacteria, fungi (Timberlake and Marshall, Science 244:1313-17, 1989), invertebrates, plants (Gasser et al., Plant Cell 1:15-24, 1989), and animals (Pursel et al., Science 244:1281-88, 1989).

Unique restriction enzyme digestion sites can be included in the nucleic acid constructs encoding antigenic components (e.g., HA proteins). These facilitate addition/deletion of epitopes, as well as the shuttling of antigenic component-encoding cassettes between a number of DNA vectors, including DNA vaccine constructs (e.g., pVax-1, Invitrogen, Carlsbad, Calif.), eukaryotic yeast expression vectors (e.g., pYes, Invitrogen, Carlsbad, Calif.), and multi-cell type expression vectors (e.g., pTriEX-4, Novagen, Madison, Wis.). This enables the production of both a DNA based immunogenic composition and vaccine, and ready production of recombinant antigenic components, which can be used directly as part of an immunogenic composition or as a protein boost. The antigenic component-encoding sequences (e.g., those encoding an HA protein or antigenic portion thereof) can also be incorporated into attenuated viral vectors such as modified vaccinia or adenovirus to serve as a boosting agent.

The term “whole viruses” in reference to vectors includes, for example, live attenuated viruses and replication-incompetent viruses. Methods of viral attenuation are well know in the art, and include, but are not limited to, high serial passage (e.g., in susceptible host cells under specific environmental conditions to select for attenuated virions), exposure to a mutagenic agent (e.g., a chemical mutagen or radiation), genetic engineering using recombinant DNA technology (e.g., using gene replacement or gene knockout to disable one or more viral genes), or some combination thereof. As is well known to one of skill in the art, replication-incompetent viruses lack the ability to cause disease, yet retain the ability to infect and deliver desired nucleotide sequences to cells. Elimination of the disease-causing potential is normally achieved by deleting a subset of genetic elements from the viral genome to prevent independent viral replication in a subject. To manufacture these vectors, they are commonly propagated in producer cells (or packaging cells) engineered to complement the replication-incompetent virus by expressing the subset of genetic elements deleted from the viral genome. A number of animal viruses have been employed as replication-incompetent vectors, including, for example, adenoviruses, adeno-associated viruses, retroviruses, lentiviruses, herpesviruses, poxviruses, alphaviruses, and picornaviruses.

The term “viral vector” refers to a nucleic acid molecule (e.g., a plasmid) that includes virus-derived nucleic acid elements that typically facilitate transfer or integration of the nucleic acid molecule. As will be evident to one of ordinary skill in the art, viral vectors can include various viral components in addition to nucleic acid(s). Sources of viral vectors include, for example, adenoviruses, adeno-associated viruses, retroviruses, lentiviruses, herpesviruses, poxviruses, alphaviruses, and picornaviruses.

Nucleic acid molecules encoding an antigenic component (e.g., an HA polypeptide) can be operably linked to regulatory sequences or elements. Regulatory sequences or elements include, but are not limited to promoters, enhancers, transcription terminators, a start codon (e.g., ATG), stop codons, and the like. As used herein, “operably linked” refers to a relationship between two nucleic acid sequences where the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid sequence is directed by an operably linked processing sequence; the translation of a nucleic acid sequence is directed by an operably linked translational regulatory sequence; the transport or localization of a nucleic acid sequence or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence.

Transformation of a host cell with a vector carrying a nucleic acid molecule encoding an antigenic component (e.g., an HA polypeptide) can be carried out by conventional techniques, as are well known to those skilled in the art. By way of example, where the host is prokaryotic, such as E. coli, competent cells that are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, methods of transfection of DNA, such as calcium phosphate coprecipitates, conventional mechanical procedures such as microinjection, electroporation, insertion of a plasmid encased in liposomes, or virus vectors, may be used. Eukaryotic cells can also be cotransformed with influenza virus nucleic acid molecules, and a second foreign DNA molecule encoding a selectable phenotype, such as the herpes simplex thymidine kinase gene. Another method is to use a eukaryotic viral vector, such as simian virus 40 or bovine papilloma virus, to transiently infect or transform eukaryotic cells and express the protein (see, e.g., Eukaryotic Viral Vectors, Cold Spring Harbor Laboratory, Gluzman ed., 1982).

In some embodiments, the immunogenic compositions described herein further include a pharmaceutically acceptable carrier and/or an adjuvant. The pharmaceutically acceptable carriers useful in this invention are conventional. Remington's Pharmaceutical Sciences (18^{th} ed.; Mack Publishing Company, Eaton, Pa., 1990), describes compositions and formulations suitable for pharmaceutical delivery of one or more antigenic components, such as one or more HA- or HA fragment-encoding nucleic acid molecules, HA-expressing vectors, and/or HA proteins combined with various pharmaceutically acceptable additives, as well as a dispersion base or vehicle. Desired additives include, but are not limited to, pH control agents, such as arginine, sodium hydroxide, glycine, hydrochloric acid, citric acid, and the like. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), reducing agents (e.g., glutathione), and preservatives (e.g., antimicrobials, and antioxidants) can be included. In general, the nature of the carrier will depend on the particular mode of administration being employed.

Various adjuvants may be used to increase the immunological response to the immunogenic compositions described herein. By “adjuvant” is intended a compound or mixture that enhances the immune response to an antigen. An adjuvant can serve as a tissue depot that slowly releases the antigen, and also as a lymphoid system activator that non-specifically enhances the immune response. Often, a primary challenge with an antigen alone, in the absence of an adjuvant, will fail to elicit a humoral or cellular immune response. Adjuvants include, but are not limited to, complete Freund's adjuvant, incomplete Freund's adjuvant, lipopolysaccharide and lipopolysaccharide derivatives (e.g., MPL™, 3-O-deacylated monophosphoryl lipid A; Corixa, Hamilton Ind.), saponin and saponin derivatives (e.g., QS21), mineral gels such as aluminum hydroxide (e.g., Amphogel™, Wyeth Laboratories, Madison, N.J.), surface active substances such as lysolecithin, pluronic polyols, polyanions, peptides, oil or hydrocarbon emulsions, keyhole limpet hemocyanins, dinitrophenol, CpG oligonucleotides, BCG (bacille Calmette-Guerin, the attenuated Mycobacterium bovis bacterium) and Corynebacterium parvum. Development of vaccine adjuvants for use in humans is reviewed in Singh et al. (Nat. Biotechnol. 17:1075-1081, 1999). Preferably, the adjuvant is pharmaceutically acceptable.

The immunogenic compositions described herein can be dispersed in a base or vehicle, which can include a hydrophilic compound having a capacity to disperse an immunogenic composition and any desired additives. The base can be selected from a wide range of suitable compounds, including but not limited to, copolymers of polycarboxylic acids or salts thereof, carboxylic anhydrides (e.g., maleic anhydride) with other monomers (e.g., methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including, fluid or viscous solutions, gels, pastes, powders, microspheres and films for direct application to a mucosal surface.

The immunogenic compositions can be combined with the base or vehicle according to a variety of methods, and release of the immunogenic compositions can be by diffusion, disintegration of the vehicle, or associated formation of water channels. In some circumstances, the immunogenic compositions are dispersed in microcapsules (microspheres) or nanocapsules (nanospheres) prepared from a suitable polymer, for example, isobutyl 2-cyanoacrylate (see, e.g., Michael et al., J. Pharmacy Pharmacol. 43:1-5, 1991), and dispersed in a biocompatible dispersing medium, which yields sustained delivery and biological activity over a protracted time. The immunogenic compositions of the invention can alternatively contain as pharmaceutically acceptable vehicles substances required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

In certain embodiments, the immunogenic compositions described herein can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the invention can be brought about by including in the composition agents that delay absorption, for example, aluminum monostearate hydrogels and gelatin. When controlled release formulations are desired, controlled release binders suitable for use in accordance with the invention include any biocompatible controlled release material which is inert to the active agent and which is capable of incorporating the immunogenic compositions described herein. Numerous such materials are known in the art. Useful controlled-release binders are materials that are metabolized slowly under physiological conditions following their delivery (e.g., at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present invention include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (e.g., L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, e.g., Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

In another embodiment, the present invention pertains to a method for making a multi-valent influenza vaccine, including (i) determining the range of antigenic diversity among an antigenic component within a target population of influenza virus strains; (ii) selecting the antigenic component from four or more influenza viruses representative of the antigenic diversity of the target population of influenza virus strains, where cross-protective immunity to the antigenic component provides immunity to multiple viruses within the target population; and (iii) preparing an immunogenic composition having at least one vector capable of expressing the selected antigenic components, where the vector includes regulatory sequences operably linked to antigenic component-encoding sequences. In a further embodiment, the immunogenic composition includes four or more vectors, each capable of expressing a single antigenic component. In specific, non-limiting examples, the antigenic component includes HA proteins, or antigenic portions thereof.

The influenza virus antigenic components contemplated herein include antigenic components (e.g., viral transmembrane and surface proteins and glycoproteins, such as HA and NA proteins and antigenic portions/fragments thereof) encoded by any influenza virus of interest. Fusion proteins are also contemplated that include a heterologous amino acid sequence chemically linked to an antigenic component. Exemplary heterologous sequences include short amino acid sequence tags (such as six histidine residues), as well a fusion of other proteins (such as c-myc or green fluorescent protein fusions). Epitopes of the antigenic components that are recognized by an antibody or that bind the major histocompatibility complex, and can be used to induce a specific immune response, are also contemplated. By “epitope” is intended an antigenic determinant. These are particular chemical groups or contiguous or non-contiguous peptide sequences on a molecule that are antigenic, that is, that elicit a specific immune response. An antibody binds a particular antigenic epitope based on the three dimensional structure of the antibody and the matching (or cognate) epitope.

As used herein, the term “antibodies” includes intact immunoglobulins as well as a number of well-characterized fragments. For instance, Fabs, Fvs, and single-chain Fvs (SCFvs) that bind to target protein (or epitope within a protein or fusion protein) would also be specific binding agents for that protein (or epitope). These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody, a genetically engineered molecule containing the variable region of the light chain, the variable region of the heavy chain, linked by a suitable polypeptide linker as a genetically fused single chain molecule. Methods of making these fragments are routine (see, for example, Harlow and Lane, Using Antibodies: A Laboratory Manual, CSHL, New York, 1999). Antibodies for use in the methods of this invention can be monoclonal or polyclonal.

Methods for expressing large amounts of protein from a cloned gene introduced into E. coli may be utilized for the purification and functional analysis of proteins. For example, fusion proteins consisting of amino terminal peptides encoded by a portion of the E. coli lacZ or trpE gene linked to an HA protein can be used to prepare polyclonal and monoclonal antibodies against this protein.

Intact native protein may also be produced in E. coli in large amounts for functional studies. Methods and plasmid vectors for producing fusion proteins and intact native proteins in bacteria are described by Sambrook et al. (ed.), Molecular Cloning: A Laboratory Manual, 2^{nd} ed., vol. 1-3, Cold Spring Harbor Laboratory Press, Cold Spring Harbor, N.Y., 1989. Such fusion proteins may be made in large amounts, are easy to purify, and can be used to elicit antibody response. Native proteins can be produced in bacteria by placing a strong, regulated promoter and an efficient ribosome-binding site upstream of the cloned gene. If low levels of protein are produced, additional steps may be taken to increase protein production; if high levels of protein are produced, purification is relatively easy. Suitable methods are presented by Sambrook et al. (ed.), Molecular Cloning: A Laboratory Manual, 2^{nd} ed., vol. 1-3, Cold Spring Harbor Laboratory Press, Cold Spring Harbor, N.Y., 1989, and are well known in the art. Often, proteins expressed at high levels are found in insoluble inclusion bodies. Methods for extracting proteins from these aggregates are described by Sambrook et al. (ed.), Molecular Cloning: A Laboratory Manual, 2^{nd} ed., vol. 1-3, Cold Spring Harbor Laboratory Press, Cold Spring Harbor, N.Y., 1989.

Isolation and purification of recombinantly expressed proteins may be carried out by conventional means including preparative chromatography and immunological separations. Additionally, the proteins can be chemically synthesized by any of a number of manual or automated methods of synthesis known in the art.

The term “antigenic portion” (e.g., of an antigenic component, such as an HA protein) includes any part, piece, or segment that is representative of, or derived from, a whole and retains the antigenic characteristics (e.g., the ability to elicit a specific immune response, such as a cell-mediated immune response, a humoral immune response, or both) of the whole. As used herein, “antigenic diversity” in reference to a target population of influenza virus strains refers to the variability of antigens (e.g., HA- or NA-derived) expressed by a population of influenza viruses. By “target population” is intended a set of influenza viruses relevant to a particular group of subjects for whom protective immunity is desired, and includes viruses of a particular subtype (e.g., H3N2, H1N1, H5N1, H9N2, H6N1, or H7N7), type (e.g., influenza A or B viruses), geographic region (i.e., a mixture of types and subtypes), or all influenza viruses that infect a particular subject (e.g., humans).

Exemplary methods for determining the range of antigenic diversity among an antigenic component within a target population of influenza virus strains include, but are not limited to, determining the amino acid sequence for at least a portion of the antigenic regions of the antigenic component (e.g., by sequencing DNA or cDNA derived from viral RNA encoding the same), antigenic analysis of the antigenic component (e.g., hemagglutination-inhibition, microneutralization, or enzyme-linked immunosorbent assay), and in vivo testing in an animal or human subject.

Sequencing the antigenic regions of antigenic components (e.g., HA proteins) from a target population of influenza viruses enables the construction of phylogenetic trees, which facilitate a visual display of the relationships between viruses or portions of viruses. Viruses which have a similar sequence will be on the same “branch” of the tree; the closer two viruses are to one another, the more related they are. Viruses tend to cluster together in the tree in groupings called “clades.” Often these clades represent antigenically distinct groups of viruses. Viruses within a clade are similar, while viruses in different clades are less similar. Methods of protein sequencing are well known in the art (e.g., Edman degradation). As is well known in the art, the sequence of a protein (e.g., an HA or NA protein) can also be deduced from the mRNA or gene encoding the protein.

Antigenic analysis of antigenic components (e.g., HA proteins) can be performed by a number of methodologies known to one of ordinary skill in the art, such as, for example, microneutralization (see, e.g., Casals, J. Immunological techniques for animal viruses in Methods in Virology. Volume III. Maramorosch, K. and Koprowski, H. (eds.), Academic Press, NY, 113-94, 1967; Zielinska et al., Virology J. 2:84, 2005), enzyme-linked immunosorbent assay (ELISA; Engvall, Meth. Enzymol., 70:419-39, 1980) and derivatives thereof, and hemagglutination-inhibition.

Neutralization assays can be used to detect the presence of antibodies directed against a specific antigenic component in a preparation (e.g., serum) or to characterize an antigenic component using antibodies to probe its antigenicity. Viruses such as influenza will undergo replication in certain cell lines (e.g., Madin-Darby canine kidney (MDCK) cells) producing viral proteins, viral particles and cytopathic effects on the cell monolayer. In a neutralization assay, a preparation that may contain virus expressing the antigenic component that is being examined is mixed with a preparation that may contain antibody that may recognize that antigenic component. If antibodies present in the mixture can bind in sufficient number and to sufficiently important antigenic regions as to prevent replication of the virus in the cells, then production of viral proteins, whole viruses and generation of cytopathic effects do not occur. The amount of virus or antibody in the initial preparations can be estimated by titration, and the specificity of the antibody or the antigenic component can be determined by incubation with multiple partners.

Detection of neutralization can be done by numerous methods to detect the virus, viral proteins or cytopathic effect. In the case of the standard microneutralization assay, the assay is performed in 96 well plates suitable for use in a microplate reader such as that used for ELISA. After mixture of the antibody containing and antigen containing preparations, and incubation on the cells for a period of time sufficient to induce the desired effect (e.g., 18-22 hours), the cells are washed, and antibody specific for the nucleoprotein of influenza viruses is added as a primary antibody. A secondary antibody conjugated to a colorimetric, fluorescent or luminescent substrate is added, and light or change in color is detected using an ELISA plate reader.

Enzyme immunoassays such as ELISA can be readily adapted to accomplish antigenic analysis of antigenic components (e.g., HA proteins) according to the methods of this invention. As understood by one of skill in the art, enzyme immunoassays can be used either for antigenic analysis of the HA by incubating unknown viruses with known antibodies, or for characterization of antibodies from unknown sera using known viruses. In the methodology of determining what viruses are in a population, the former would be appropriate; for evaluating the immune response, the latter would be appropriate.

An ELISA method effective for the detection of soluble antigenic components is the direct competitive ELISA. This method is most useful when a specific antigenic component antibody (e.g., an HA-specific antibody) and purified antigenic component (e.g., an HA antigen) are available. Briefly: 1) coat a substrate (e.g., a microtiter plate) with a sample suspected of containing an antigenic component; 2) contact the bound antigenic component with an antigenic component-specific antibody bound to a detectable moiety (e.g., horseradish peroxidase enzyme or alkaline phosphatase enzyme); 3) add purified inhibitor antigenic component; 4) contact the above with the substrate for the enzyme; and 5) observe/measure inhibition of color change or fluorescence and quantitate antigenic component concentration (e.g., using a microtiter plate reader).

An additional ELISA method effective for the detection of soluble antigenic components is the antibody-sandwich ELISA. This method is frequently more sensitive in detecting antigen than the direct competitive ELISA method. Briefly: 1) coat a substrate (e.g., a microtiter plate) with an antigenic component-specific antibody; 2) contact the bound antigenic component antibody with a sample suspected of containing an antigenic component; 3) contact the above with antigenic component-specific antibody bound to a detectable moiety (e.g., horseradish peroxidase enzyme or alkaline phosphatase enzyme); 4) contact the above with the substrate for the enzyme; and 5) observe/measure color change or fluorescence and quantitate antigenic component concentration (e.g., using a microtiter plate reader).

An ELISA method effective for the detection of cell-surface antigenic components is the direct cellular ELISA. Briefly, cells suspected of exhibiting a cell-surface antigenic component are fixed (e.g., using glutaraldehyde) and incubated with an antigenic component-specific antibody bound to a detectable moiety (e.g., horseradish peroxidase enzyme or alkaline phosphatase enzyme). Following a wash to remove unbound antibody, substrate for the enzyme is added and color change or fluorescence is observed/measured.

In the hemagglutination-inhibition (HI) assay, sera from subjects (e.g., mice, ferrets or humans) who have been exposed to different strains of influenza virus is mixed with the same virus strains. Influenza viruses will agglutinate (i.e., bind in solution) red blood cells, causing them to stay in suspension rather than form a visible pellet, in a process termed hemagglutination. Antibodies binding to antigenic components (e.g., HA proteins and/or NA proteins) on the viruses will inhibit hemagglutination in a concentration-dependent manner. High affinity antibody directed against a particular HA antigen can typically inhibit hemagglutination despite being diluted significantly (e.g., after a 1 to 2056 dilution). Antibodies directed at a similar related HA antigen can still bind, but not as efficiently, such that fewer dilutions can be done before hemagglutination is abrogated (e.g., a dilution of 1 to 512). A four-fold difference in titer in the HI assay is typically taken as a reasonable approximation of antigenic drift sufficient to interrupt immunity. If the titer is less than 2, the antibodies cannot bind and/or cannot neutralize their target. Antigenic maps of the relationships between viruses can be constructed in this manner.

Once the antigenic diversity of an antigenic component of a target population of influenza viruses is known, viruses from within that population are selected, such that cross-protective immunity to the selected set of viruses provides immunity in a subject to multiple viruses within the target population. As used herein, the term “cross-protective immunity” refers to the generation of protective immunity in a subject to multiple influenza virus strains of a target population of viruses following administration of a therapeutically effective amount of an immunogenic composition disclosed herein. While not being bound by any theory, it is believed that although different influenza virus strains possess different antigenic component repertoires (e.g., different HA and NA antigens), some of the protective antigens are shared among heterologous serotypes, and expression of these shared antigens in a subject can lead to cross-protective immunity to multiple virus strains within the target population.

If the antigenic diversity of the target population is large, then many viruses are selected. If the antigenic diversity is limited, then fewer viruses are selected. There is a minimum number of viruses that are included (i.e., four), however, more viruses can be included to provide overlapping coverage of multiple strains. Thus if estimates are made based on serology data, the more viruses that are selected the less chance that any viruses will be “left out” (i.e., not covered by the selected set of antigenic components, such as the selected set of HA and/or NA antigens). These viruses can be selected using sequence maps (e.g., phylogenetic trees), antigenic maps, computer modeling techniques based on one or both of these methods, or by simpler methods, such as selecting one virus from each year that a viral type or subtype has circulated in the population. Viruses that are not yet in existence can also be included by inferring their properties either by mathematical or computer modeling, by selecting variations on the sequence of existing viruses, or by forcing the production of such viruses experimentally by infecting vaccinated or previously infected animals and assaying for escape mutants.

Methods for inducing an immune response in a subject against an influenza virus are also encompassed by the present invention, and include administering to a subject a therapeutically effective amount of the immunogenic compositions described herein (e.g., an antigenic component-expressing vector, such as an HA-expressing vector), typically combined together with one or more pharmaceutically acceptable vehicles and, optionally, other therapeutic ingredients (e.g., antibiotics, or anti-inflammatories). In some embodiments, a therapeutically effective amount of one or more viral proteins or antigenic portions thereof (e.g., HA and/or NA proteins), or one or more live attenuated viruses, derived from the target population of influenza virus strains is further administered to the subject. Such a prime:boost strategy (or coordinate vaccination protocol) is used to enhance an immune response elicited by the antigenic component-expressing vector alone (e.g., an HA-expressing vector). Typically, when a prime:boost strategy is employed, the antigenic component-expressing vector immunogen and the viral protein/live attenuated virus booster are administered coordinately, in a specified temporal sequence (e.g., separated by two weeks, three weeks, one month, three months, etc.).

Within the methods of the invention, the immunogenic compositions can be administered to subjects by a variety of mucosal administration modes, including by oral, rectal, upper respiratory tract (i.e., the nasal cavity, pharynx, larynx, and trachea), intrapulmonary, or transdermal delivery. Mucosal administration can be by way of spray, droplet, aerosol, or by topical delivery. Optionally, the immunogenic compositions can be administered by non-mucosal routes, including by intramuscular, subcutaneous, subdermal, intravenous, intra-atrial, intra-articular, intraperitoneal, or parenteral routes. In other alternative embodiments, the immunogenic compositions described herein, such as HA-expressing vectors, can be administered in a DNA vaccination method.

For genetic immunization, suitable delivery methods known to those skilled in the art include direct injection of plasmid DNA into muscles (Wolff et al., Hum. Mol. Genet. 1:363, 1992), delivery of DNA complexed with specific protein carriers (Wu et al., J. Biol. Chem. 264:16985, 1989), direct mucosal administration of DNA (Eo et al., J. Immunol. 166:5473, 2001; Makitalo et al., J. Gen. Virol. 85:2407, 2004); co-precipitation of DNA with calcium phosphate (Benvenisty and Reshef, Proc. Natl. Acad. Sci. 83:9551, 1986), encapsulation of DNA in liposomes (Kaneda et al., Science 243:375, 1989), particle bombardment (e.g., using DNA-coated gold microprojectiles) (Tang et al., Nature 356:152, 1992; Eisenbraun et al., DNA Cell Biol. 12:791, 1993), and in vivo infection using cloned retroviral vectors (Seeger et al., Proc. Natl. Acad. Sci. 81:5849, 1984). Similarly, nucleic acid vaccine preparations can be administered via viral carrier (e.g., via whole viruses, such as live attenuated influenza viruses).

The immunogenic compositions disclosed herein can be administered to the subject in a single bolus delivery, via continuous delivery (e.g., continuous transdermal, mucosal or intravenous delivery) over an extended time period, or in a repeated administration protocol (e.g., by a weekly or monthly, repeated administration protocol). The therapeutically effective dosage of the immunogenic composition can be provided as repeated doses within a prolonged prophylaxis or treatment regimen, that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, ferret, porcine, feline, non-human primate, and other accepted animal model subjects known in the art.

Alternatively, effective dosages can be determined using in vitro models (e.g., immunologic and histopathologic assays). Using such models, only ordinary calculations and adjustments are required to determine an appropriate concentration and dose to administer a therapeutically effective amount of the immunogenic composition (e.g., amounts that are effective to elicit a desired immune response or alleviate one or more symptoms of a targeted disease). In alternative embodiments, an effective amount or effective dose of the immunogenic composition may simply inhibit or enhance one or more selected biological activities correlated with a disease or condition, as set forth herein, for either therapeutic or diagnostic purposes.

The actual dosage of the immunogenic composition will vary according to factors such as the disease indication and particular status of the subject (e.g., the subject's age, size, fitness, extent of symptoms, susceptibility factors, and the like), time and route of administration, other drugs or treatments being administered concurrently, as well as the specific pharmacology of the immunogenic composition for eliciting the desired activity or biological response in the subject. Dosage regimens can be adjusted to provide an optimum prophylactic or therapeutic response. When provided prophylactically, the immunogenic compositions of the invention are provided in advance of any symptom. Prophylactic administration serves to prevent or ameliorate any subsequent infection with an influenza virus. The immunogenic compositions of the invention can thus be provided prior to the anticipated exposure to one or more influenza virus strains, so as to attenuate the anticipated severity, duration or extent of an infection and/or associated disease symptoms, after exposure or suspected exposure to the viruses, or after the actual initiation of an infection.

Upon administration of an immunogenic composition of the invention (e.g., via injection, aerosol, oral, topical or other route), the immune system of the subject typically responds to the immunogenic composition by producing antibodies specific for multiple influenza viruses within the target population. Such a response signifies that an immunologically effective dose of the immunogenic composition was delivered. An immunologically effective dosage can be achieved by single or multiple administrations. For each particular subject, specific dosage regimens can be evaluated and adjusted over time according to the individual need and professional judgment of the person administering or supervising the administration of the immunogenic composition. In some embodiments, the antibody response of a subject administered the immunogenic compositions of the disclosure will be determined in the context of evaluating effective dosages/immunization protocols. In most instances it will be sufficient to assess the antibody titer in serum or plasma obtained from the subject. Decisions as to whether to administer booster inoculations and/or to change the amount of the immunogenic composition administered to the subject can be at least partially based on the antibody titer level. The antibody titer level can be based on, for example, an immunobinding assay which measures the concentration of antibodies in the serum which bind to a specific antigen, for example, an HA or NA antigen. The ability to neutralize in vitro and in vivo biological effects of target influenza virus strains can also be assessed to determine the effectiveness of the treatment.

Typical subjects intended for treatment with the immunogenic compositions and methods of the present invention include humans, as well as non-human primates and other animals. To identify subjects for prophylaxis or treatment according to the methods of the invention, accepted screening methods are employed to determine risk factors associated with a targeted or suspected disease or condition (e.g., influenza), or to determine the status of an existing disease or condition in a subject. These screening methods include, for example, conventional work-ups to determine environmental, occupational and other such risk factors that may be associated with the targeted or suspected disease or condition, as well as diagnostic methods, such as various ELISA and other immunoassay methods, which are available and well known in the art to detect and/or characterize disease-associated markers. These and other routine methods allow the clinician to select patients in need of therapy using the methods and immunogenic compositions of the invention. In accordance with these methods and principles, an immunogenic composition can be administered according to the teachings herein as an independent prophylaxis or treatment program, or as a follow-up, adjunct or coordinate treatment regimen to other treatments, including conventional influenza vaccines.

The instant invention also includes kits, packages and multi-container units containing the herein described immunogenic compositions, active ingredients, and/or means for administering the same for use in the prevention and treatment of influenza in subjects. In one embodiment, these kits include a container or formulation that contains one or more of the immunogenic compositions and/or other active agents described herein. In one example, this component is formulated in a pharmaceutical preparation for delivery to a subject. The immunogenic compositions are optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the immunogenic composition packaged therewith can be used.

As used herein, the singular terms “a,” “an” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. All publications and patent applications are herein incorporated by reference to the same extent as if each individual publication or patent application was specifically and individually indicated to be incorporated by reference.

The subject matter of the present disclosure is further illustrated by the following non-limiting examples.

### EXPERIMENTAL

### Example 1

### A Multi-Valent H3N2 DNA Vaccine

As a first approximation of a working multiple antigen influenza vaccine, DNA vaccination in an H3N2 subtype model system was tested. The experimental design included vaccinating mice using a DNA plasmid expressing the HAs of target strains, then measuring the resulting homologous and heterologous antibody titers raised in the mice as a correlate of immunity. Mice were then challenged with homologous or heterologous virus to assay protection by determination of lung titers.

As a first step, the target population of viruses was determined to be human H3N2 viruses isolated since 1968 when the viruses entered humans. H3N2 viruses can be divided into groups based on antigenicity; eleven distinct clusters have been identified (Smith et al., Science 305:371, 2004). Mice were vaccinated against viruses from the first (HK68), third (Vic75), and fifth (Len86) of these clusters and then immunity was measured to representative strains from the first ten clusters (including two viruses from the closest clusters).

### Creation of Vector DNA Expressing Influenza HAs and Vaccination of Mice

Hemagglutinin from the human H3N2 influenza virus isolates A/Hong Kong/1/68 (HK68, GenBank accession number AF348176), A/Victoria/3/75 (VI75, GenBank accession number V01098) and A/Leningrad/6/86 (LE86, GenBank accession number DQ508849) were individually cloned into a pHW2000 plasmid vector as described by Hoffmann et al. (Proc. Natl. Acad. Sci. U.S.A 97:6108, 2000). Individual plasmids were bound to one micron gold beads and propelled onto the shaved abdomen of anesthetized mice using a gene gun. When pHW2000 was delivered as a vector control, 2.4 μg pHW2000 was bound to one milligram gold. For administration of individual HAs, 1.6 μg pHW2000 per milligram gold was mixed with 0.8 μg of the individual HA to maintain a total of 2.4 μg DNA per milligram gold. When all three HAs were delivered simultaneously, each HA was bound at a concentration of 0.8 μg per milligram gold (2.4 μg total plasmid DNA). In all instances, the individual DNA components were mixed thoroughly prior to addition to the gold particles.

### Vaccination Regimen

Each mouse was vaccinated with a total of 2.4 μg DNA on one milligram gold, as described herein. Mice that received three exposures of DNA were inoculated via the gene gun at 30-day intervals. Sera collected from mice 14 days after the third exposure to DNA were analyzed for the presence of influenza-specific IgG1 expression by ELISA.

### Enzyme-Linked Immunosorbent Assay

Egg-grown reassortant influenza viruses expressing the A/Syd/5/97 neuraminidase were concentrated, purified over a sucrose gradient and inactivated with 0.025% formalin. ELISA plates were coated with 1 μg HA mL⁻¹ in PBS. Plates were washed with PBS containing 0.05% (v/v) Tween-20 (PBST) and blocked with 10% FBS in PBST (FBS-PBST). RDE-treated sera were diluted in FBS-PBST. Alkaline phosphatase-conjugated goat anti-mouse IgG1 diluted 1:1000 in FBS-PBST, was used as a detection antibody, with p-nitrophenyl phosphate as a substrate. OD was read at 405 nm 1 hour after substrate addition. Reciprocal serum antibody titers were calculated at 50% maximal binding on the titration curve. Titers for samples with OD values less 4 times the OD measured in normal mouse serum at the starting serum dilution (1:50) were reported as having a titer of less than 50.

### Results

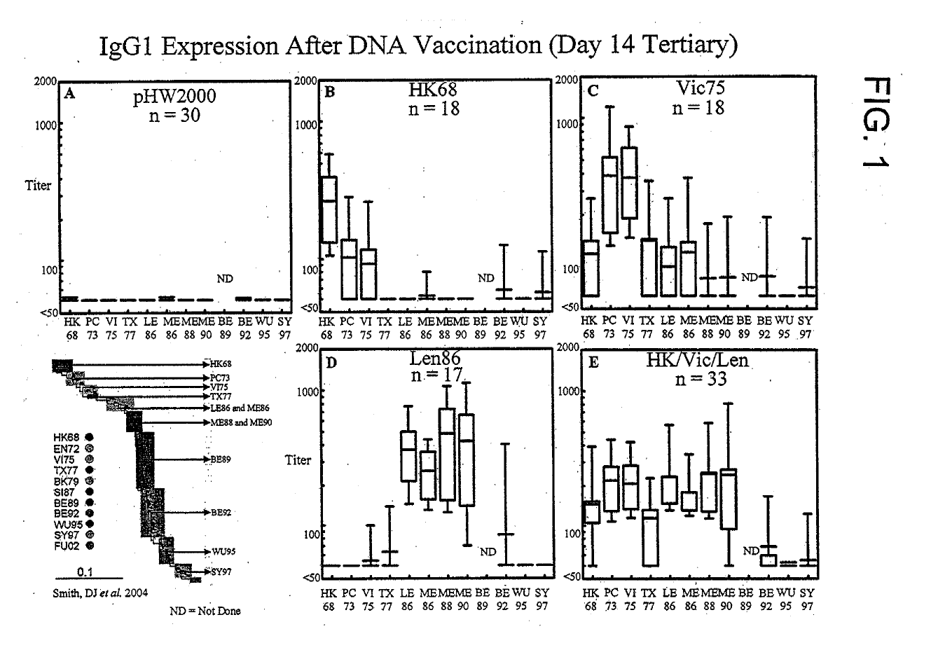

Antibodies could be generated by DNA vaccination against both homologous and related (within 2-3 antigenic clusters) heterologous strains (FIG. 1). Vaccination with multiple constructs simultaneously led to immunity against all three homologous strains as well as related strains without evidence of competition or immunodominance (FIG. 1E).

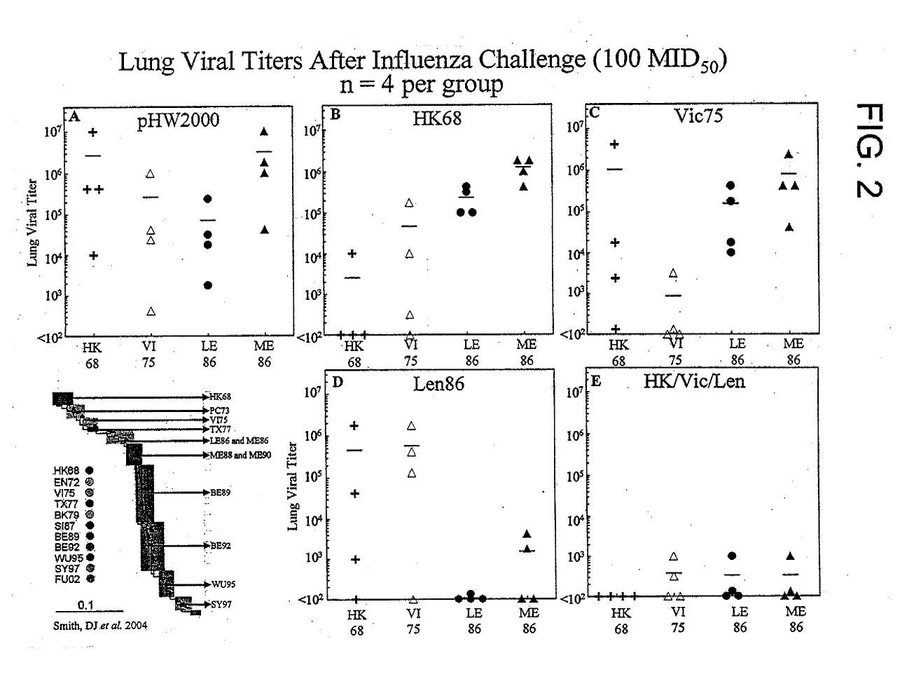

Challenge with homologous or heterologous (Mem86) viruses demonstrated that modest reductions in lung viral titer could be seen that were strongest against homologous strains (FIG. 2). No competition was observed since mice who were vaccinated against three strains simultaneously had the best outcomes, demonstrating that vaccination against multiple related viruses can generate immune responses that are similar to or superior to vaccination with each individual component alone.

### Example 2

### A Multi-Valent H3N2 Live Attenuated Virus Vaccine

The methods employed in the multi-valent H3N2 DNA vaccination regime were extended to a second method of vaccination, using a live attenuated influenza virus. Furthermore, in preparation for assessing the efficacy of live attenuated virus vaccination in concert with DNA vaccination in a prime:boost manner, mice were mock-primed with empty DNA vector (pHW2000) in these initial experiments.

### Construction of 1:1:6 Reassortant Viruses

Individual influenza genes cloned into pHW2000 plasmid vectors as described (Hoffmann et al., Proc. Natl. Acad. Sci. U.S.A 97:6108, 2000) were used to create reassortant influenza viruses. Six of the eight gene segments required to create viable influenza virus (PB1, PB2, NS, NP, PA, and M) were from the mouse-adapted laboratory strain A/Puerto Rico/8/34 (A/PR/8/34, H1N1). The HA component of these viruses was provided by the individual HAs described herein. The NA component used for creation of these viruses was from an H3N2 influenza isolate (A/Sydney/5/97). Additional HAs were incorporated into the pHW2000 plasmid as described herein, and incorporated onto the A/PR/8/34 backbone using the Sydney (N2) neuraminidase. The additional HAs used for virus creation were: A/Port Chalmers/1/73 (GenBank accession number CY009348), A/Texas/1/77 (GenBank accession number AF450246), A/Memphis/6/86 (GenBank accession number M21648), A/Memphis/8/88 (GenBank accession number CY010756), A/Memphis/7/90 (GenBank accession number CY008740), A/Beijing/353/89 (GenBank accession number AF008684), A/Beijing/32/92 (GenBank accession number AF008812), A/Wuhan/359/95 (GenBank accession number AY661190), and A/Sydney/5/97 (GenBank accession number AF180584).

As an additional vehicle for the delivery of influenza HA to mice, viruses were created as described herein, using two genes (PB1 and PB2) from A/PR/8/34 that were mutated to incorporate a temperature-sensitive (ts) phenotype on these viruses as described (Jin et al., J. Virol. 78:995, 2004). These viruses were shown to have a ts phenotype and appropriate genotype. These ts influenza viruses were administered to mice 21 days after DNA administration, and sera was collected 21 days after exposure to these viruses. To avoid interference of anti-neuraminidase immunity, all ts influenza viruses were created using neuraminidase from the A/Hong Kong/1/68 influenza isolate (H3N2).

### Vaccination Regimen

Mice that were primed with DNA and boosted with influenza virus ts mutants were inoculated with DNA on day 0, and received influenza ts mutants (10⁷ TCID₅₀ of each virus) on day 21 via the intranasal route. Serum was collected from mice 21 days after influenza ts mutant administration (secondary response) and analyzed by HI.

### Hemagglutination-Inhibition

Sera collected after exposure to influenza virus ts mutants (day 21 of the secondary response to influenza HA) was analyzed for influenza-specific antibody using a standard HI titer assay. RDE-treated sera was mixed with 4 HA units of the individual viruses and incubated with the diluted sera for 1 hour at 4° C. Chicken red blood cells (0.5%) were added to the plates and HI titers, reported as the reciprocal of the final serum dilution that inhibits hemagglutination, were determined 30 minutes later.

### Results

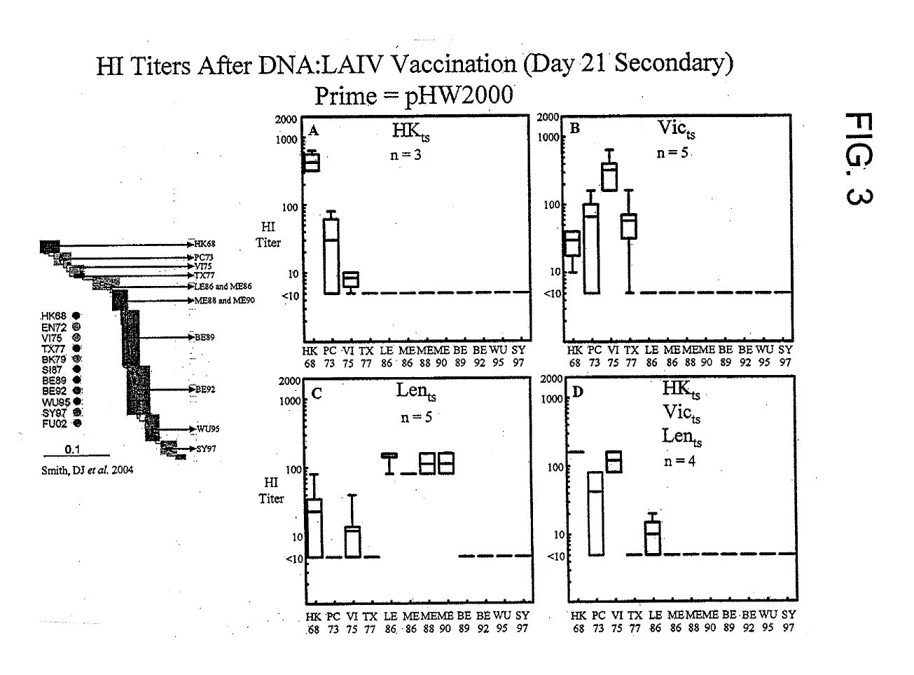

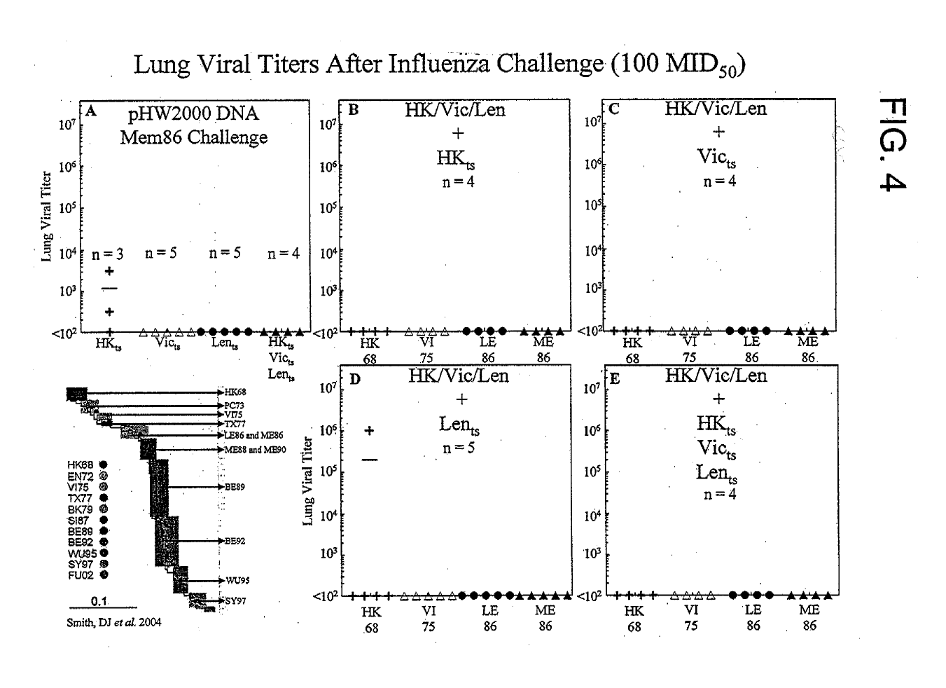

Antibody responses were generated against all three homologous strains, as well as related viruses from different antigenic clusters (FIG. 3). When all three were given simultaneously, antibody responses were generated to all three although there appeared to be some minor competition in responses since the mean antibody titer by HI to the Len86 component was lower after triple vaccination than after single vaccination. This possible competition did not affect the ability of the vaccine to induce sterilizing immunity, however, as no virus was recovered from mice challenged with Len86 after vaccination with all three strains. The immunity generated by this method was sufficient to prevent replication of a heterologous strain, Mem86 in single antigen vaccination regimens including Vic75 or Len86 as the target antigen and with the multiple antigen vaccination (FIG. 4A). Viral replication was seen in all mock-vaccinated mice.

### Example 3

### Prime:boost Vaccination Using a DNA Immunogen and Live Attenuated Virus Boost

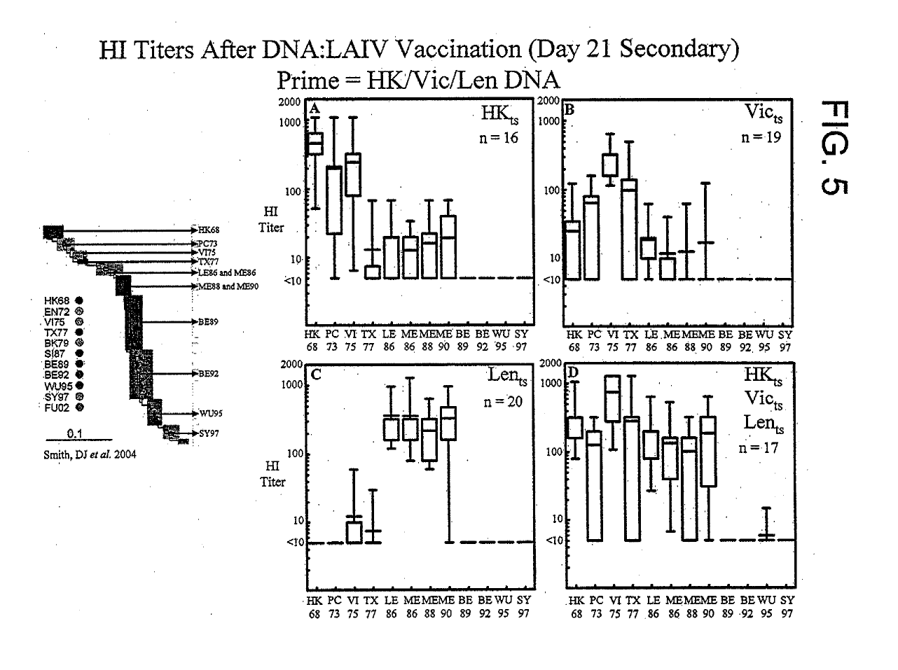

Both methods, DNA and live attenuated viruses, were tested in a prime:boost regimen. Priming with all three HA targets (HK/Vic/Len) using DNA followed by vaccination with each one alone (HKₜₛ, Vicₜₛ or Lenₜₛ), or all three together (HKₜₛ/Vicₜₛ/Lenₜₛ), induced strong, cross-protective immunity (FIG. 5). The effect of the DNA priming was to broaden and strengthen the quality of the antibody responses to the live attenuated vaccine (compare FIG. 3D with FIG. 5D). The prime:boost vaccination regimen induced sterilizing immunity against all three homologous strains as well as the heterologous strain Mem86 (FIG. 4).

### Example 4

### A Universal Influenza Immunization Protocol for Children

At three-months of age, an intramuscular DNA vaccine is administered. The vaccine includes vectors (e.g., plasmids) coding for 20 hemagglutinin proteins from H3N2 viruses, 15 hemagglutinin proteins from H1N1 viruses, and 10 hemagglutinin proteins from influenza B viruses. At six-months of age, a cocktail of 45 live attenuated influenza viruses is administered intranasally. The 45 live attenuated influenza viruses express the same 45 hemagglutinin proteins, with 15 different neuraminidase proteins. At one-year of age a protein boost that contains 2 micrograms of DNA from each of the 45 influenza virus strains is administered intramuscularly. Immunity to these 45 strains provides not only specific immunity to the matching strains, should they circulate, but related strains (e.g., those in the target population and/or those that arise due to antigenic drift or reassortment).

### Example 5

### A Universal Influenza Immunization Protocol Against H5N1 Viruses

A cocktail of replication-incompetent Sindbis viruses is administered intranasally. The replication-incompetent Sindbis viruses code for 50 hemagglutinin proteins from H5N1 avian influenza strains, including all major H5N1 strains that have ever been identified and a number of theoretical strains that can be generated in the laboratory or using computer modeling. Three-weeks following the initial vaccination, a boost with the exact same preparation is administered. This prime:boost protocol provides immunity to all H5N1 viruses that have the potential to emerge from birds.

1. An immunogenic composition, comprising at least one vector capable of expressing four or more hemagglutinin proteins, or antigenic portions thereof, representative of the antigenic diversity of a target population of influenza virus strains, wherein said composition elicits an immune response in a subject to said target population of influenza virus strains.

2. The immunogenic composition of claim 1, wherein said vector comprises a plasmid.

3. The immunogenic composition of claim 1, wherein said vector comprises a viral vector.

4. The immunogenic composition of claim 1, wherein said vector comprises a whole virus.

5. The immunogenic composition of claim 4, wherein said whole virus comprises a live attenuated virus.

6. The immunogenic composition of claim 4, wherein said whole virus comprises a replication-incompetent virus.

7. The immunogenic composition of claim 1, wherein said target population of influenza virus strains comprises influenza viruses of an individual type.

8. The immunogenic composition of claim 1, wherein said target population of influenza virus strains comprises influenza viruses of an individual subtype.

9. The immunogenic composition of claim 1, wherein said target population of influenza virus strains comprises influenza viruses of a geographic region.

10. The immunogenic composition of claim 1, wherein said hemagglutinin proteins, or antigenic portions thereof, are selected from the group consisting of type B influenza, subtype H3N2 type A influenza, subtype H1N1 type A influenza, subtype H5N1 type A influenza, subtype H9N2 type A influenza, subtype H6N1 type A influenza, subtype H7N7 type A influenza, and combinations thereof.

11. The immunogenic composition of claim 1, further comprising at least one of a pharmaceutically acceptable carrier or an adjuvant.

12. An immunogenic composition, comprising at least four vectors, each vector capable of expressing a different hemagglutinin protein, or antigenic portion thereof, representative of the antigenic diversity of a target population of influenza virus strains, wherein said composition elicits an immune response in a subject to said target population of influenza virus strains.

13. The immunogenic composition of claim 12, wherein said vectors comprise plasmids.

14. The immunogenic composition of claim 12, wherein said vectors comprise viral vectors.

15. The immunogenic composition of claim 12, wherein said vectors comprise whole viruses.

16. The immunogenic composition of claim 15, wherein said whole viruses comprise live attenuated viruses.

17. The immunogenic composition of claim 15, wherein said whole viruses comprise replication-incompetent viruses.

18. The immunogenic composition of claim 12, wherein said target population of influenza virus strains comprises influenza viruses of an individual type.

19. The immunogenic composition of claim 12, wherein said target population of influenza virus strains comprises influenza viruses of an individual subtype.

20. The immunogenic composition of claim 12, wherein said target population of influenza virus strains comprises influenza viruses of a geographic region.

21. The immunogenic composition of claim 12, wherein said hemagglutinin protein, or antigenic portion thereof, is selected from the group consisting of type B influenza, subtype H3N2 type A influenza, subtype H1N1 type A influenza, subtype H5N1 type A influenza, subtype H9N2 type A influenza, subtype H6N1 type A influenza, subtype H7N7 type A influenza, and combinations thereof.

22. The immunogenic composition of claim 12, further comprising at least one of a pharmaceutically acceptable carrier or an adjuvant.

23. A method for making a multi-valent influenza vaccine, comprising:
(a) determining the range of antigenic diversity among hemagglutinin proteins within a target population of influenza virus strains;
(b) selecting four or more hemagglutinin proteins representative of the antigenic diversity of said target population of influenza virus strains, wherein cross-protective immunity to said hemagglutinin proteins provides immunity to multiple viruses within said target population; and
(c) preparing an immunogenic composition comprising at least one vector capable of expressing said hemagglutinin proteins, wherein said vector comprises regulatory sequences operably linked to hemagglutinin protein encoding sequences.

24. The method of claim 23, wherein determining the range of antigenic diversity among hemagglutinin proteins within a target population of influenza virus strains comprises determining the amino acid sequence for at least a portion of the antigenic regions of said hemagglutinin proteins.

25. The method of claim 24, wherein the amino acid sequence is determined by sequencing DNA encoding at least a portion of the antigenic regions of said hemagglutinin proteins.

26. The method of claim 23, wherein determining the range of antigenic diversity among hemagglutinin proteins within a target population of influenza virus strains comprises antigenic analysis of said hemagglutinin proteins.

27. The method of claim 26, wherein said antigenic analysis comprises hemagglutination-inhibition, microneutralization, enzyme-linked immunosorbent assay, or a combination thereof.

28. The method of claim 23, wherein said vector comprises a plasmid.

29. The method of claim 23, wherein said vector comprises a viral vector.

30. The method of claim 23, wherein said vector comprises a whole virus.

31. The method of claim 30, wherein said whole virus comprises a live attenuated virus.

32. The method of claim 30, wherein said whole virus comprises a replication-incompetent virus.

33. A method for making a multi-valent influenza vaccine, comprising:
(a) determining the range of antigenic diversity among hemagglutinin proteins within a target population of influenza virus strains;
(b) selecting four or more hemagglutinin proteins representative of the antigenic diversity of said target population of influenza virus strains, wherein cross-protective immunity to said hemagglutinin proteins provides immunity to multiple viruses within said target population; and
(c) preparing an immunogenic composition comprising four or more vectors capable of expressing said hemagglutinin proteins, wherein said vectors comprise a regulatory sequence operably linked to a single hemagglutinin protein encoding sequence.

34. The method of claim 33, wherein determining the range of antigenic diversity among hemagglutinin proteins within a target population of influenza virus strains comprises determining the amino acid sequence for at least a portion of the antigenic regions of said hemagglutinin proteins.

35. The method of claim 34, wherein the amino acid sequence is determined by sequencing DNA encoding at least a portion of the antigenic regions of said hemagglutinin proteins.

36. The method of claim 33, wherein determining the range of antigenic diversity among hemagglutinin proteins within a target population of influenza virus strains comprises antigenic analysis of said hemagglutinin proteins.

37. The method of claim 36, wherein said antigenic analysis comprises hemagglutination-inhibition, microneutralization, enzyme-linked immunosorbent assay, or a combination thereof.

38. The method of claim 33, wherein said vectors comprise plasmids.

39. The method of claim 33, wherein said vectors comprise viral vectors.

40. The method of claim 33, wherein said vectors comprise whole viruses.

41. The method of claim 40, wherein said whole viruses comprise live attenuated viruses.

42. The method of claim 40, wherein said whole viruses comprise replication-incompetent viruses.

43. A method for inducing an immune response in a subject against an influenza virus, comprising administering to the subject a therapeutically effective amount of the immunogenic composition of claim 1, effective for inducing said immune response against influenza.

44. The method of claim 43, further comprising administering to said subject a therapeutically effective amount of one or more hemagglutinin proteins, or antigenic portions thereof, derived from said target population of influenza virus strains.

45. The method of claim 43, further comprising administering to said subject a therapeutically effective amount of one or more live attenuated viruses derived from said target population of influenza virus strains.

46. The method of claim 43, wherein said immunogenic composition is administered intramuscularly, subcutaneously, subdermally, or topically.

47. The method of claim 43, wherein said immunogenic composition is administered to the upper respiratory tract.

48. The method of claim 47, wherein said immunogenic composition is administered by spray, droplet, aerosol, or topically.

49. The method of claim 43, wherein a humoral or cellular immune response is induced.
